(19) United States
(12) Patent Application Publication
(10) Pub. No.: US 2007/0023285 A1
(43) Pub. Date: Feb. 1, 2007

(54) Three-dimensional bioelectrochemical sensor strip structure

(75) Inventors: Paul Yang, Chung Ho City (TW); Jen-Chien Chien, Yilan County (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD., CHUNG HO CITY (TW)

(21) Appl. No.: 11/192,065

(22) Filed: Jul. 29, 2005

(51) Int. Cl. G01N 33/487

(52) U.S. Cl. 204/403.01

(57) ABSTRACT

A three-dimensional bioelectrochemical sensor strip structure includes a pair of insulating substrates, a transparent reacting area disposed at an end of the substrates, an opening disposed around the periphery of the reacting area, an electrode having an operating electrode and a reference electrode of different polarities disposed between the substrates, a spacer disposed between the electrodes, and a hydrophilic enzyme covered onto an end of the electrode facing the substrate. When a test is performed, an end of the electrode not covered by the hydrophilic enzyme is inserted into the sensing machine, and the testing object (such as blood) is dripped into any position of the opening of the reacting area, such that the enzyme is dissolved quickly. With the spacer, the electrodes at both sides of the spacer provide a guide of a channel, and a dedicated biochemical reaction is conducted between the oxidation-reduction substance and the test object to produce an electron transfer. The concentration of the testing object can be detected by the sensing machine. The hydrophilic enzyme on the electrode allows the testing object to be absorbed into the reacting area easily, and the direction of the opening of the reacting area is not restricted, and thus facilitating the testing object to enter into the reacting area.

### BACKGROUND OF THE INVENTION

1. Field of the Invention

The present invention relates to a three-dimensional bioelectrochemical sensor strip structure that uses an oxidation-reduction substance and a testing object for a dedicated biochemical reaction to produce an electron transfer, and the strip structure goes with a sensor to detect the concentration of the testing object. The reacting area of the strip structure is comprehensive, such that the testing object can enter into the reacting area much easier without a limitation of directions.

2. Description of the Related Art

Since the concept of household nursing becomes increasingly popular, and there are various different kinds of sensor products for a fast, cheap, non-professional operation, and these sensor products include a blood sugar meter, an electronic ear thermometer, and an electronic sphygmomanometer, wherein a disposable blood sugar testing paper used for the blood sugar meter makes use of the principle of the bioelectrochemical sensor strip to detect the blood sugar concentration of the testing object.

At present, there are various kinds of blood sugar testing paper products in the market, and the application of the testing object is mainly divided into a dripping type and a quantitative siphoning type. The design of the dripping type testing paper usually comes with a plastic grid covered onto the reacting area for preventing contaminations, but the required quantity of the testing object is generally larger than 3 μl. For senior citizens or diabetics who have to take a blood sample by a needle, it is very inconvenient for these people to drip a drop of blood precisely into the reacting area in order to provide sufficient amount of blood for the test. As a result, many pieces of testing paper may be wasted due to improper operations.

On the other hand, the siphoning type testing paper only requires a blood sample of less than 3 μl. If a small amount of testing object is in contact with an absorbing opening, the testing object will be absorbed into the reacting area. However, the design of the siphoning type testing paper sold in the market comes with an excessively small size that causes inconvenient access and contaminates the absorbing opening easily. Furthermore, there is only one absorbing opening installed with a fixed direction, which will make the operation inconvenient. The testing paper of this sort requires a blood sample of less than 3 μl, and such result is achieved by reducing the area of an electrode in the reacting area. However, reducing the area also makes the signal very weak, and it is necessary to use a more precise instrument to facilitate the signal detection.

Referring to FIGS. 1 and 2, a dripping type sheet electrode testing paper comprises an electrically insulating substrate having an anode-cathode system thereon and being covered on the electrode system to define a reacting area and an anode-cathode connector connected to a sensor; a reacting film being spread in the reacting area and a plastic grid being covered on the reacting area, and finally a tape being stuck on an exposed area of the reacting area for fixing the plastic grid in position. However, the method of applying the testing object for the testing paper of this sort requires users to drip a blood sample onto the reacting area. Furthermore, the required quantity of testing object can only be dropped to 9 μl.

Referring to FIGS. 3 and 4 for R.O.C. Pat. No. 573735 entitled a blood sugar testing strip structure, the strip structure comprises:

a substrate;

a first electrode, being disposed on the a first substrate and having an end as an electrode connecting point and the other end as an electrode enzyme acting area;

an insulating layer, being covered onto the first substrate and having an opening disposed at the electrode enzyme reacting are;

an enzyme film, being coated onto the electrode enzyme reacting area at an opening of the insulating layer; and

a second substrate, being covered onto the utmost top layer of the blood sugar testing strip structure and at the bottom of the second substrate including:

a second electrode, having an end as an electrode connecting point and the other end as an electrode enzyme reacting area.

The insulating layer of the blood sugar testing strip structure also forms a crevice to facilitate the siphon action, and an opening proximate to an end of the crevice as a ventilation hole for the siphon action.

Although such structure can solve the foregoing problem, yet the opening of the structure is fixed at a specific direction. The area is reduced correspondingly, and thus creating a limitation on the direction and the range of dropping the testing object. Further, this structure has to form an opening proximate to an end of the crevice as a ventilation hole for the siphon action, and thus making the application and manufacture more difficult.

### SUMMARY OF THE INVENTION

In view of the shortcomings of the prior art, the inventor of the present invention based on years of experience to conduct extensive researches and experiments to find a way to overcome the shortcomings in hope of finding a feasible solution, and finally invented the three-dimensional bioelectrochemical sensor strip structure in accordance with the present invention.

To achieve the foregoing objective, a three-dimensional bioelectrochemical sensor strip structure of the invention comprises:

a pair of electrically insulating substrates, having an adhesive on the corresponding surfaces, and being hydrophilic at a section without the adhesive for adhering and defining the size of a reacting area, and the reacting area being in a transparent form;

a spacer, disposed at the middle of the corresponding substrates to form a sandwich structure, and each side of the spacer has an electrode, and these electrodes comprise an operating electrode and a reference electrode;

a hydrophilic reacting film, including a hydrophilic enzyme, an electronic medium, an interface activating agent, and a buffer solution mixed into an even solution to be coated into the space of the reacting area for one time and covered onto the operating electrode and the reference electrode. The reacting film is in contact with the testing object to produce a dedicated biochemical reaction.

In a test, the corresponding substrates clamp a space to define a three-dimensional structure. Since the periphery of the reacting area is an opening, therefore the testing can be absorbed into the reacting area easily without being limited by the direction of the opening. Users can take sample easily. Unlike the dripping type or the siphoning type, the testing object is introduced and spread into the reacting area through the hydrophilic reacting film for the biochemical reaction.

Further, the required quantity of the testing object is less than 3 μl, and thus patients can reduce their pain of extracting blood. Since the required quantity is constant, therefore measuring error caused by different quantity of testing object for each operation can be eliminated.

Another objective of the present invention is to provide a three-dimensional bioelectrochemical sensor strip structure having an arc protruded structure opening as the edge of its opening, not only can be identified easily, but also can absorb the testing object easily, so as to prevent a dose hesitation produced when the testing object from enters into the reacting area.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a schematic view of a prior art device;

FIG. 2 is a schematic view of the use of a prior art device;

FIG. 3 is an exploded view of another prior art device;

FIG. 4 is a perspective view of another prior art device;

FIG. 5 is a perspective view of the present invention;

FIG. 6 is an exploded view of the present invention;

FIG. 6A is a schematic view of a reacting film being covered onto an electrode according to the present invention;

FIG. 7 is a planar view of the assembled structure of the present invention;

FIG. 8 is a cross-sectional view of the present invention; and

FIG. 9 is a cross-section view of the present invention when it is in use.

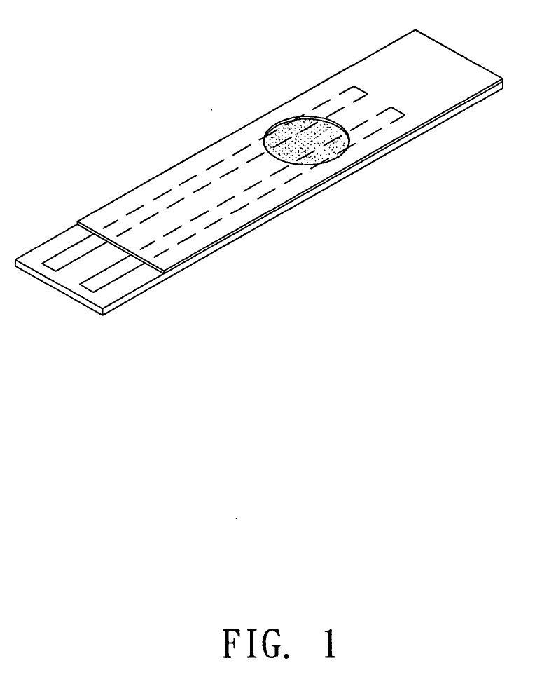

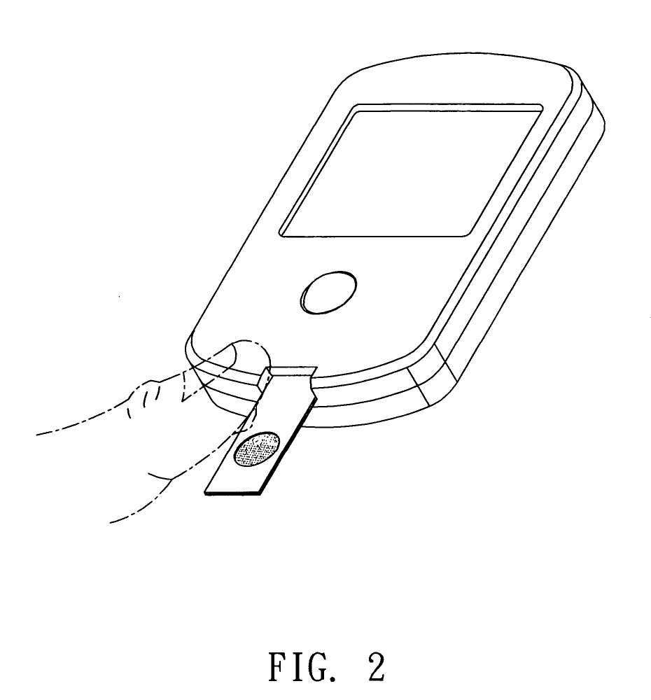

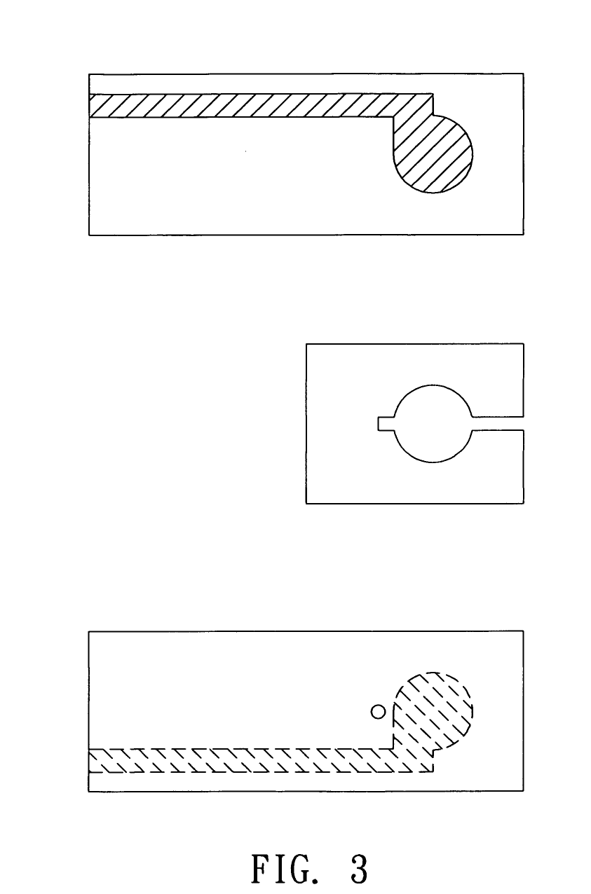

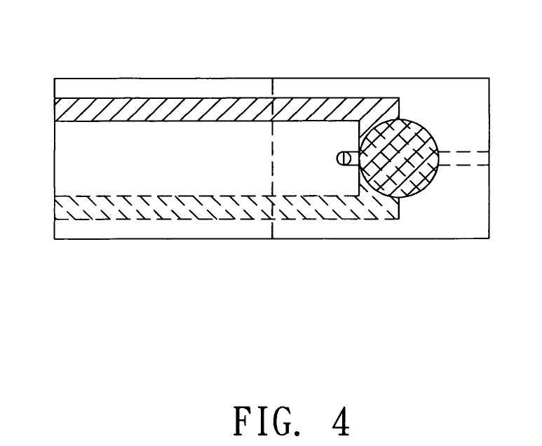

### DETAILED DESCRIPTION OF THE PREFERRED EMBODIMENTS

The present invention discloses a three-dimensional bioelectrochemical sensor strip structure that uses an oxidation-reduction substance and a testing object for a dedicated biochemical reaction to produce an electron transfer, and the strip structure goes with a sensor to dedicatedly and sensitively detect the concentration of the testing object.

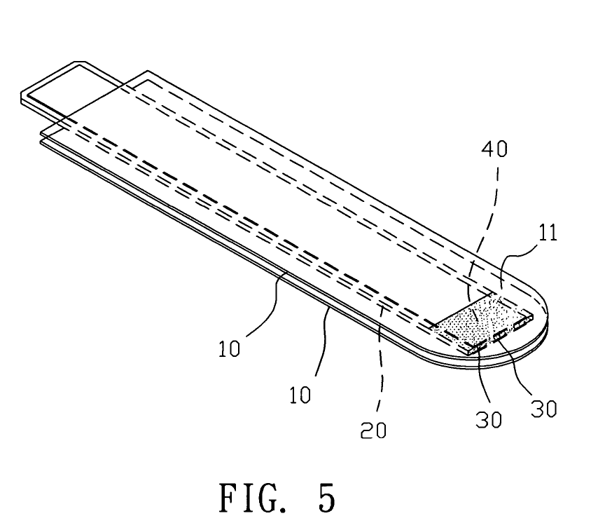

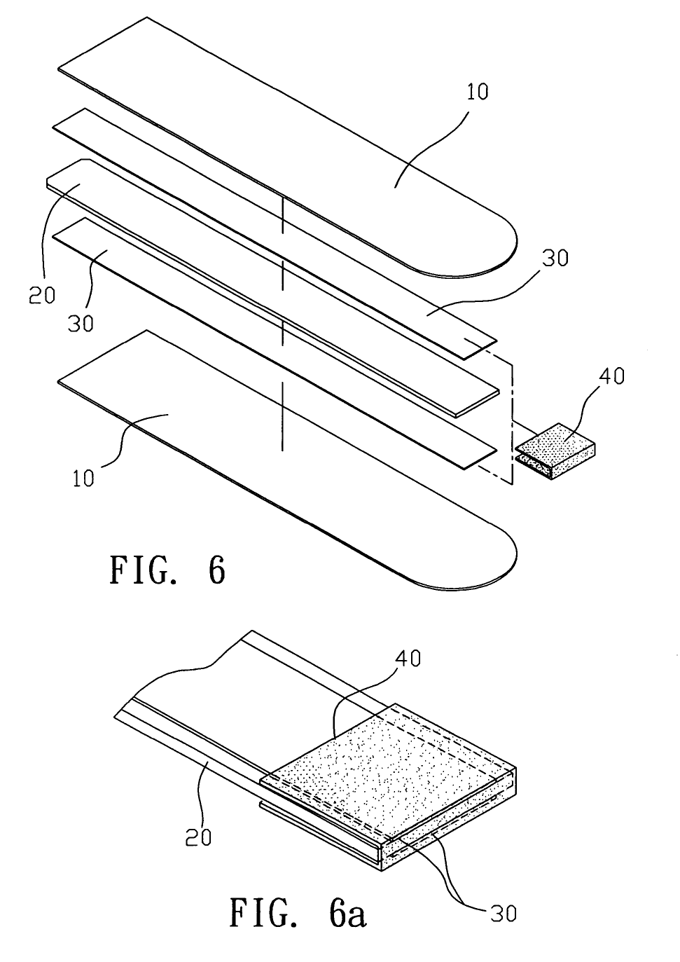

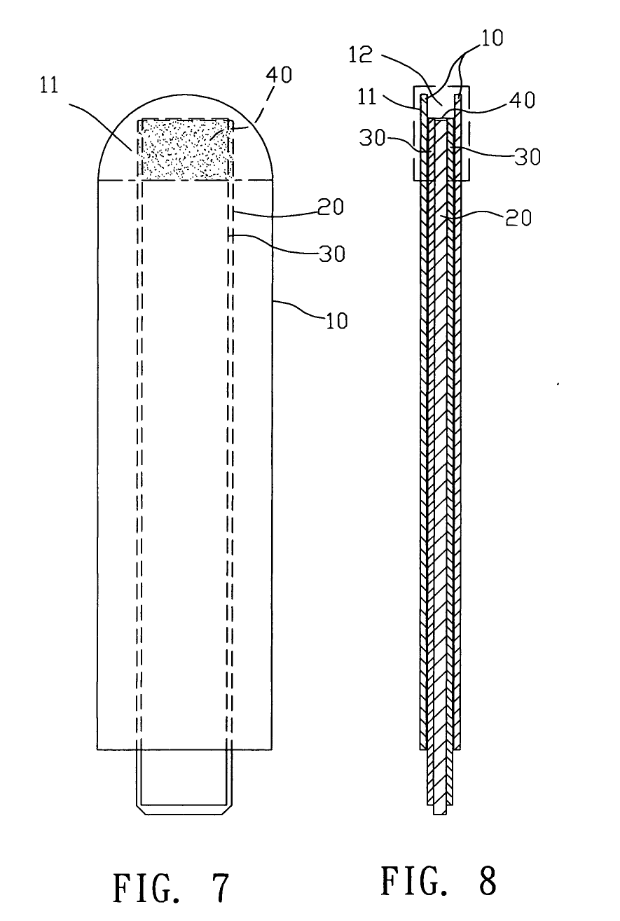

Referring to FIGS. 5, 6, 6A, 7 and 8, the strip structure comprises corresponding substrates 10 being flat in shape and electrically insulating, and the substrates 10 have an adhesive on their corresponding sides, and the section without adhesive is hydrophilic for adhering a reacting area 11 and defining the size of the reacting area. The reacting area 11 is transparent, and the periphery of the reacting area 11 is in an arc shape, and also have an opening 12. The edge of the opening 12 is an arc protruded structure, not only can be identified easily, but also can absorb and spread the testing object easily, so as to reduce the dose hesitation when the testing object enters into the reacting area 11 as shown in FIG. 8.

Further, a spacer 20 is disposed between the substrates 10, such that the spacer 20 and the substrates 10 form a sandwich structure, and one end of the spacer 20 is extended out of the substrate 10, and both sides of the spacer 20 have an electrode 30. The electrodes 30 comprises an operating electrode and a reference electrode, and one end of the electrodes 30 is extended all the way to the reacting area 11 and the other end is protruded from another end of the substrates 10.

Further, the electrodes 30 are covered by a hydrophilic reacting film 40, and the hydrophilic reacting film 40 is disposed in the space of the reacting area 11 and covers the operating electrode and reference electrode. In this embodiment, the hydrophilic reacting film 40 is a hydrophilic enzyme comprised of a formula of an oxidation-reduction substance, a hydrophilic polymer, an electronic medium, an interface activating agent, and a buffer solution for being in contact with the testing object to produce a biochemical reaction with the testing object.

The hydrophilic polymer is provided for stabilizing the oxidation-reduction substance and the electronic medium. The formula for this hydrophilic polymer and the reacting film is of 0.1-5% by weight. The usable hydrophilic polymers include hydroxypropyl methylcellulose, starch, dextran, polyacrylamide, 2-hydroxyethylmethacrylate, gelatin, chitin, albumni, polyvinylpyrrolidone (PVP), polyviny alcohol (PVA), polyvinyl acetate (PVAc), methylcellose, and carboxymethyl cellose, etc.

Another composite of the hydrophilic reacting film is an electronic medium used for a dedicated biochemical reaction between the oxidation-reduction substance and the testing object to donate and receive an electron. The electron is transferred by contacting the operating electrode and the reference electrode of the electrode system with the reacting film to an anode connector and a cathode connector on the other end of the electrode system. If the electrode 30 of a substrate 10 protruded from a bioelectrochemical sensor strip structure is connected to a sensing machine, the sensing machine can be set up to supply an external constant voltage to the strip structure and receive the foregoing electric effect by a signal receiver. A display device converts the signal into a display of the concentration of the testing object.

Another composite of the hydrophilic reacting film is an interface activating agent used for spreading and covering the reactants onto the hydrophilic reacting area, and the composition of the interface activating agent is below 0.25% by weight of the formula. The suitable interface activating agent includes but not limited to fatty acid-Acco Emulsifier, sodium laury sulfate, glycerol-monostearate, Tween 20 (polyoxyethylene sorbitan monolaurate), Triton X-100 (t-octylphenoxypolyethoxyethanol), polyoxyethylene glycerine, fatty acid, and phosphatidyl holine.

Another composite of the hydrophilic reacting film is a buffer solution which could be potassium cyanide phosphate, potassium phosphate monobasic, tri-formaldehyde aminomethane (Tris), borate, or citric acid, and deionized water. The composition of this buffer solution is 72.25˜98.8% by weight of the reactants. The buffer solution is provided for maintaining the best activation of the enzyme in the whole reaction when the buffer solution is in contact with the testing object.

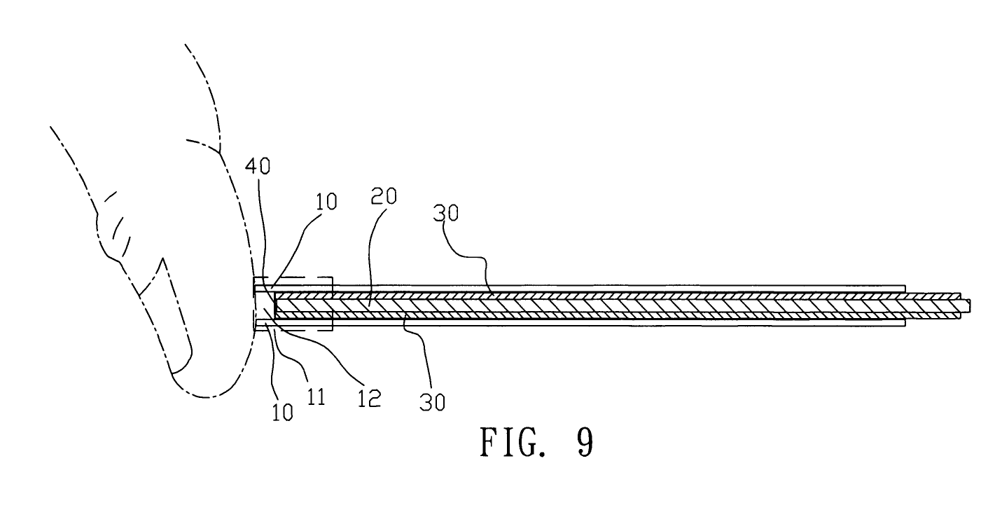

Refer to FIG. 9 for the application. When the test is preformed, a spacer 20 is clamped by the corresponding substrates 10 to produce a three-dimensional structure. Since the periphery of the reacting area 11 is an opening 12, such that the testing object can be spread and absorbed into the reacting area 11 without being limited by the direction of the opening 12 and thus facilitating taking the sample. Further, since the testing object is introduced and dispersed from the hydrophilic reacting film 40 into the reacting area 11 for a biochemical reaction, which differs from the dripping or siphoning type.

Further, the required quantity of the testing object according to the present invention is less than 3 μl, and thus it can reduce the patient's pain for extracting blood. The required quantity of the testing object is constant, and thus the measuring error caused by different operations can be reduced.

In summation of the above description, the present invention herein enhances the performance and overcomes the shortcoming of the prior art, and further complies with the patent application requirements.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A three-dimensional bioelectrochemical sensor strip structure for detecting the content of a biochemical matters in a testing object, comprising:
a pair of electrically insulating substrates, having an adhesive disposed on the corresponding sides, and the section without said adhesive being hydrophilic in property, for adhering a reacting area and defining the size of said reacting area, and said reacting area being transparent;
a spacer, disposed between said substrates to define a sandwich structure, and having an electrode disposed separately on both sides of said spacer; and
a hydrophilic reacting film, formed at the space of said reacting area where said electrode resides, and said reacting film being in contact with said testing object to produce a dedicated biochemical reaction.

2. The three-dimensional bioelectrochemnical sensor strip structure of claim 1, wherein said electrode comprises an operating electrode and a reference electrode respectively disposed on both sides of said spacer.

3. The three-dimensional bioelectrochemical sensor strip structure of claim 1, wherein said reacting film is composed of an oxidation-reduction enzyme, a hydrophilic polymer, an electronic medium, an interface activating agent and a buffer solution salt.

4. The three-dimensional bioelectrochemical sensor strip structure of claim 1, wherein said opening has an edge in the form of an arc protruded structure for providing an easy identification and absorbing in said testing object to reduce the dose hesitation when said testing object enters into said reacting area.
